(19) United States
(12) Patent Application Publication
(10) Pub. No.: US 2009/0068718 A1
(43) Pub. Date: Mar. 12, 2009

(54) AMINO ACID DERIVATIVES OF INDOLINONE BASED PROTEIN KINASE INHIBITORS

(76) Inventors: Congxin Liang, Palm Beach Gardens, FL (US); Yangbo Feng, Palm Beach Gardens, FL (US)

Correspondence Address: SCHWEGMAN, LUNDBERG & WOESSNER, P.A., P.O. BOX 2938, MINNEAPOLIS, MN, 55402, US

(21) Appl. No.: 12/159,579

(22) PCT Filed: Dec. 28, 2006
(86) PCT No.: PCT/US2006/049406

§ 371 Date: Oct. 10, 2008

Related U.S. Application Data
(60) Provisional application No. 60/754,835, filed on Dec. 29, 2005.

(51) Int. Cl. C12N 9/99; C07D 405/06; C07D 413/14

(52) U.S. Cl. 435/184; 548/468; 544/144; 544/82

(57) ABSTRACT

Amino acid derivatives of pyrrolyl-indolinones and their amide or ester derivatives have enhanced and unexpected drug properties as inhibitors of protein kinases and are useful in treating disorders related to abnormal protein kinase activities such as cancer.

### FIELD OF INVENTION

The invention relates to protein kinase inhibitors and to their use in treating disorders related to abnormal protein kinase activities such as cancer and inflammation. More particularly, the invention relates to amino acid derivatives of pyrrolyl-indolinones and their amide or ester derivatives and their pharmaceutically acceptable salts employable as protein kinase inhibitors.

### BACKGROUND

Protein kinases are enzymes that catalyze the phosphorylation of hydroxyl groups of tyrosine, serine, and threonine residues of proteins. Many aspects of cell life (for example, cell growth, differentiation, proliferation, cell cycle and survival) depend on protein kinase activities. Furthermore, abnormal protein kinase activity has been related to a host of disorders such as cancer and inflammation. Therefore, considerable effort has been directed to identifying ways to modulate protein kinase activities. In particular, many attempts have been made to identify small molecules that act as protein kinase inhibitors.

Several pyrrolyl-indolinone derivatives have demonstrated excellent activity as inhibitors of protein kinases (Larid et al. FASEB J. 16, 681, 2002; Smolich et al. Blood, 97, 1413, 2001; Mendel et al. Clinical Cancer Res. 9, 327, 2003; Sun et al. J. Med. Chem. 46, 1116, 2003). The clinical utility of these compounds has been promising, but has been partially compromised due to the relatively poor aqueous solubility and/or other drug properties. What is needed is a class of modified pyrrolyl-indolinone derivatives having both inhibitory activity and enhanced drug properties.

### SUMMARY

One aspect of the invention is directed to a compound having the following structure represented by Formula I:

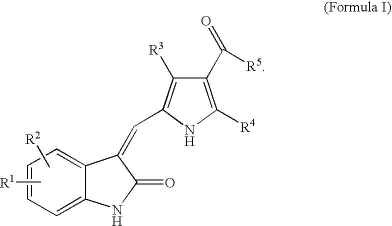

In Formula I, R¹ is selected from the group consisting of hydrogen, halo, (C1-C6) alkyl (C3-C8) qycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl; R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino; R³ is selected from the group consisting of hydrogen, (C1-C6) alkyl, (C6-C10) aryl, (C5-C10) heteroaryl, and amide; R⁴ is selected from the group consisting of hydrogen and (C1-C6) alkyl; and R⁵ is an alpha or beta amino acid or an alpha or beta amino amide group connected to the carbonyl of (I) through the alpha or beta amino group to form an amide bond; or a pharmaceutically acceptable salt or prodrug thereof or it may act as a prodrug. In a preferred embodiment, R⁵ is represented by the following structure:

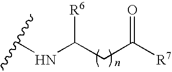

In the above structure, R⁶ is a side chain of a naturally or unnaturally occurring amino acid or its corresponding amide derivative thereof, the amide derivative having an amide nitrogen represented by NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids; R⁷ is selected from the group consisting of hydroxy, (C1-C6) O-alkyl, (C3-C8) O-cycloalkyl, and by NR⁸R⁹; and n is 0 or 1. In a first subgenus, R⁵ is an alpha amino acid where the alpha amino group is connected to the carbonyl of Formula I to form an amide bond. Preferred species within the first subgenus are represented by the following structures:

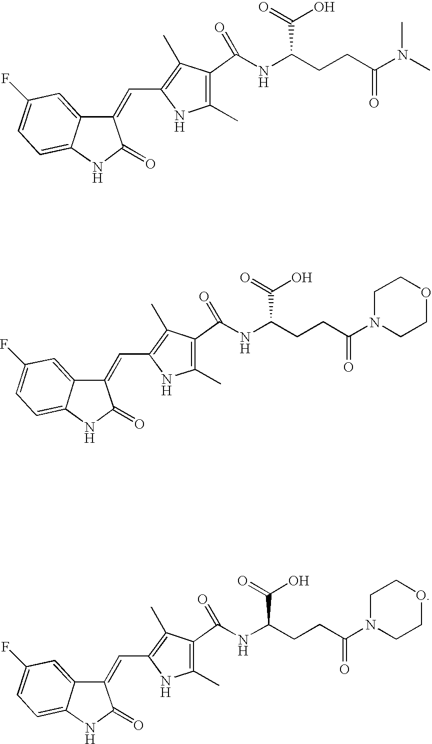

In a second subgenus, R⁵ is an alpha amino amide where the alpha amino group is connected to the carbonyl of Formula I to form an amide bond. Preferred species within the second subgenus are represented by the following structures:

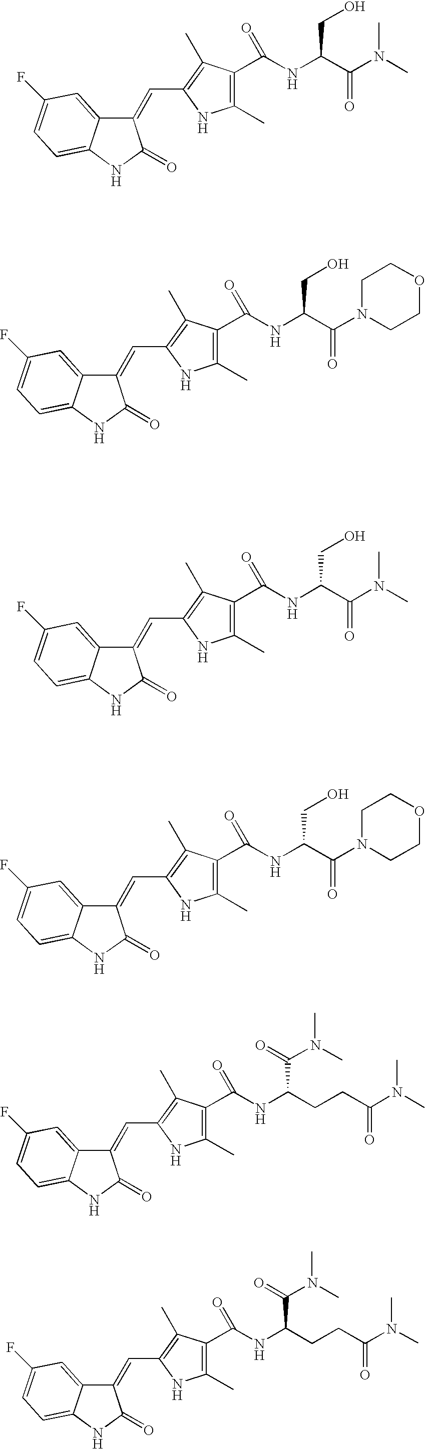

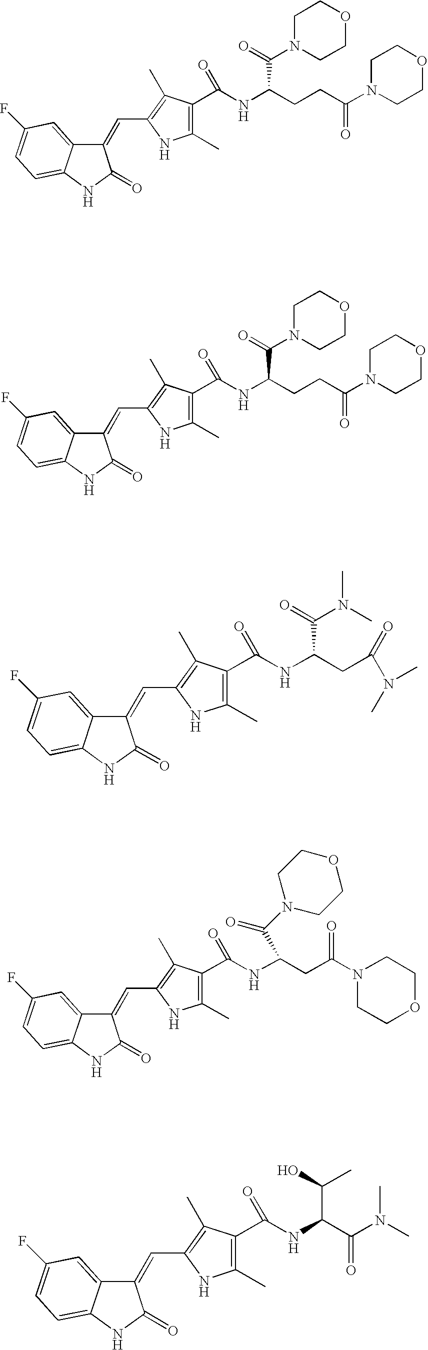

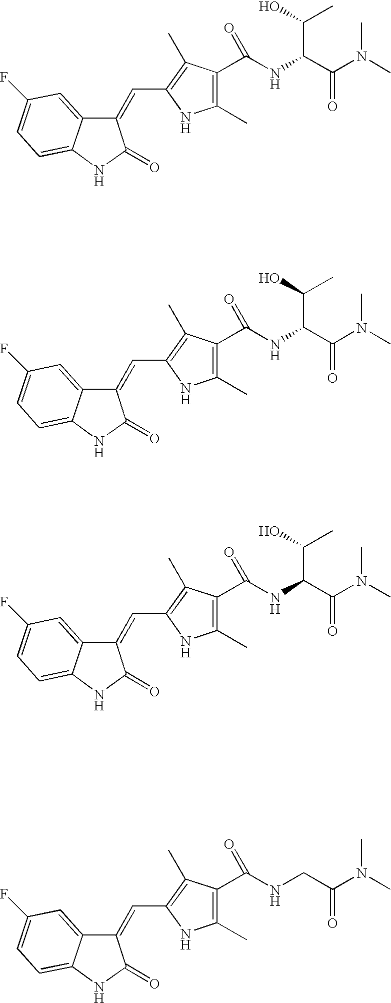

In a third subgenus, R⁵ is a beta amino acid where the beta amino group is connected to the carbonyl of Formula I to form an amide bond. A preferred species within the third subgenus is represented by the following structure:

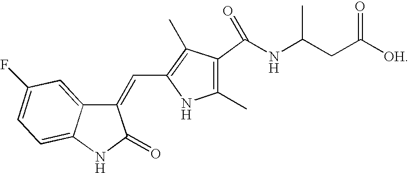

In a fourth subgenus, R⁵ is a beta amino amide where the beta amino group is connected to the carbonyl of Formula I to form an amide bond. Preferred species within the fourth subgenus are represented by the following structures:

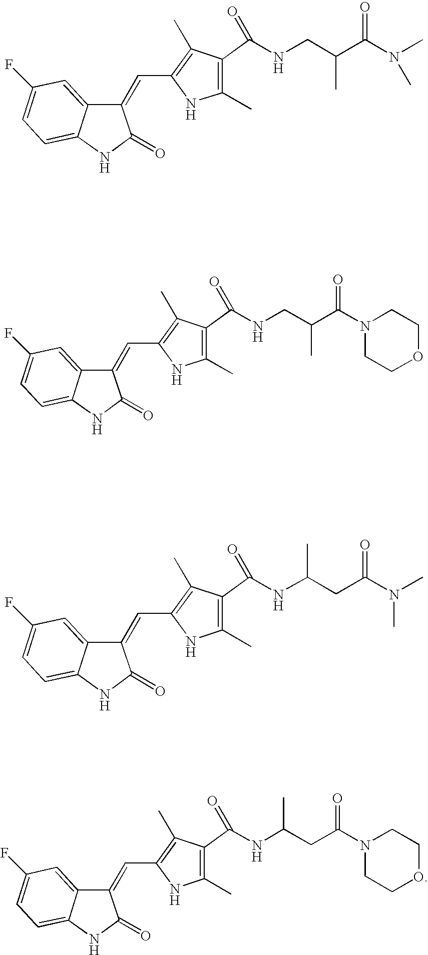

Another aspect of the invention is directed to a method for the modulation of the catalytic activity of a protein kinase with a compound or salt of Formula I. In a preferred mode, the protein kinase is selected from the group of receptors consisting of VEGF, and PDGF.

### Utility:

The present invention provides compounds capable of regulating and/or modulating protein kinase activities of, but not limited to, VEGFR and/or PDGFR. Thus, the present invention provides a therapeutic approach to the treatment of disorders related to the abnormal functioning of these kinases. Such disorders include, but are not limited to, solid tumors such as glioblastoma, melanoma, and Kaposi's sarcoma, and ovarian, lung, prostate, pancreatic, colon and epidermoid carcinoma. In addition, VEGFR/PDGFR inhibitors may also be used in the treatment of restenosis and diabetic retinopathy.

Furthermore, this invention relates to the inhibition of vasculogenesis and angiogenesis by receptor-mediated pathways, including the pathways comprising VEGF receptors, and/or PDGF receptors. Thus the present invention provides therapeutic approaches to the treatment of cancer and other diseases which involve the uncontrolled formation of blood vessels.

### Synthetic Protocol:

A general scheme for synthesizing the starting material HATU ester (1-1) is shown in Scheme 1.

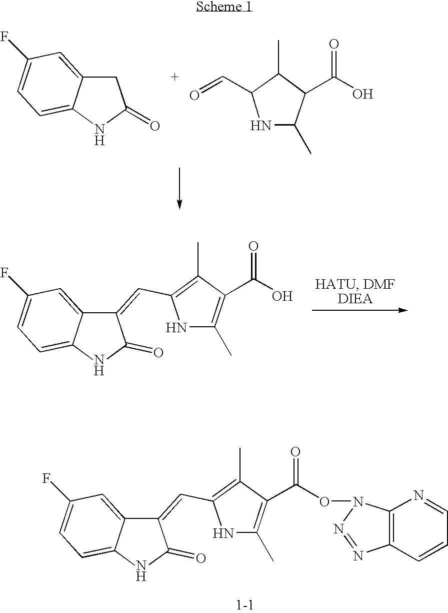

### Step 1:

A mixture of 5-fluoro-1, 3-dihydroindol-2-one (1.62 g, 10.2 mmol), 5-formyl-2,4-dimethyl-1H-pyrrole-3-carboxylic acid (1.96 g, 10.7 mmol), pyrrolidine (12 drops) and absolute ethanol was heated to reflux for 3 hours. The mixture was cooled to 25° C. and the solids were collected by filtration. The solids were stirred with ethanol (30 mL) at 72° C. for 30min. The mixture was cooled to 25° C. and the solids were collected again by filtration, washed with ethanol (6 mL), and dried under vacuum overnight to give an orange solid (Z)-5-((5-fluoro-2-oxoindolin-3-ylidene)methyl)-2,4-dimethyl-1H-pyrrole-3-carboxylic acid (3.094 g, 96%). LC-ESIMS observed [M+H]⁺ 301 (calculated for C₁₆H₁₃FN₂O₃ 300.09).

### Step 2:

(Z)-5-((5-fluoro-2-oxoindolin-3-ylidene)methyl)-2,4-dimethyl-1H-pyrrole-3-carboxylic acid (3.094 g, 10.3 mmol) was suspended in DMF (15 mL), and stirred for 5 minutes. DIEA (2.7 mL, 15.5 mmol) was then added and the mixture was stirred for 10 minutes. HATU (3.91 g, 10.28 mmol) was added and the reaction mixture was stirred at 25° C. for completion. LC/MS detected the completion of the reaction. Most of the DMF was removed and the residue was suspended in ACN and stirred for another 40 minutes. The solid was collected by filtration, washed with ACN, and dried under high vacuum overnight. (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (3.97 g, 92%) was obtained. LC-ESIMS observed [M+H]⁺ 419 (calculated for C₂₁H₁₅FN₆O₃ 418.12).

### EXAMPLES 1-23

### The general scheme

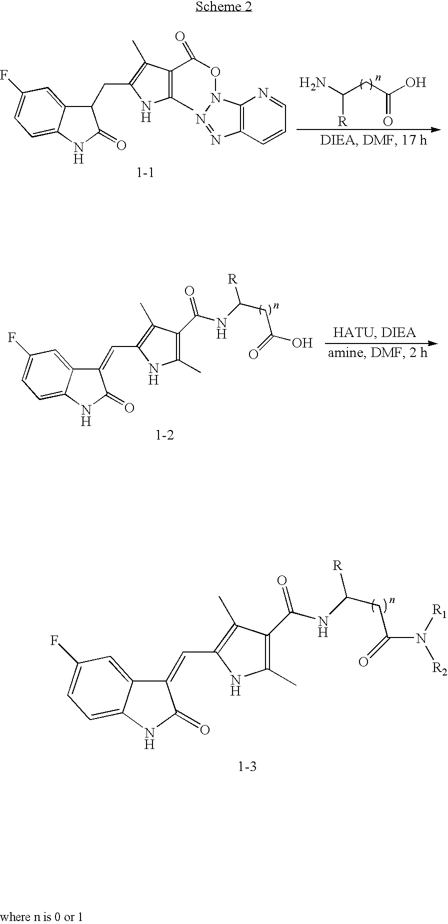

The synthesis of the starting material HATU ester (1-1) is shown in Scheme 1. To prepare the free carboxylic acid 1-2, the unprotected amino acid (1.0 equiv) was added to a solution of 1-1 (1.0 equiv) and DIEA (1.5 equiv) in DMF, as shown in Scheme 2. After stirring the solution at 25° C. overnight, LC-MS indicated that the formation of 1-2 was complete, and no starting materials remained. This solution was directly used in the next step to prepare the amide 1-3. Thus, an amine (2 equiv), HATU (1.0 mmol), and DIEA (1 equiv) were added to the solution. After stirring for 2 h at 25° C., the reaction was complete based on LC-MS analysis. The reaction solution was directly subjected to preparative HPLC to obtain the pure amide product 1-3, which was subsequently characterized by LC-MS and NMR spectroscopy.

### EXAMPLE 1

### Preparation of 5-[5-Fluoro-2-oxo-1,2-dihydro-indol-(3Z)-ylidenemethyl]-2,4-dimethyl-1H-pyrrole-3-carboxylic acid (2-dimethylcarbamoyl-propyl)-amide

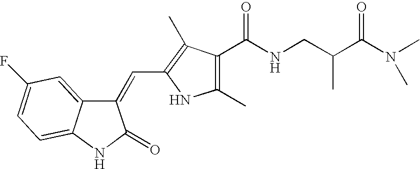

Preparative HPLC gave 50 mg of the title compound (96%) from 52 mg starting material (the active ester 1-1). LC-MS: single peak at 254 nm, MH⁺ calcd. for C₂₂H₂₅FN₄O₃: 413, obtained: 413. ¹H-NMR (DMSO-d_{6,} 400 MHz), δ 13.68 (s, 1H), 10.89 (s, 1H), 7.76 (dd, J=2.4 Hz, 9.6 Hz, 1H), 7.71 (s, 1H), 7.68 (t, J=5.6 Hz, 1H), 6.93 (m, 1H), 6.84 (dd, J=4.4 Hz, 8.4 Hz, 1H), 3.31 (m, 1H), 3.16 (m, 2H), 3.05 (s, 3H), 2.84 (s, 3H), 2.41 (s, 3H), 2.39 (s, 3H), 1.03 (d, J=6.8 Hz, 3H).

### EXAMPLE 2

### 5-[5-Fluoro-2-oxo-1,2-dihydro-indol-(3Z)-ylidenemethyl]-2,4-dimethyl-1H-pyrrole-3-carboxylic acid (2-methyl-3-(morpholin4-yl)-3-oxo-propyl)-amide

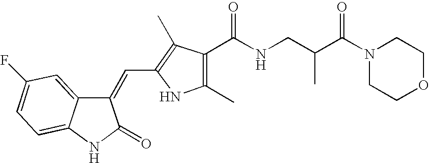

Preparative HPLC gave 56 mg of the title compound (98%) from 52 mg starting material (the active ester). LC-MS: single peak at 254 nm, MH⁺ calcd. for C₂₄H₂₇F₂N₄O₄: 455, obtained: 455. ¹H-NMR (DMSO-d₆, 400 MHz), δ 13.68 (s, 1H), 10.89 (s, 1H), 7.75 (dd, J=2.4 Hz, 9.2 Hz, 1H), 7.71 (s, 1H), 7.67 (t, J=5.6 Hz, 1H), 6.92 (m, 1H), 6.83 (dd, J=4.8 Hz, 8.4 Hz, 1H), 3.55 (m, 7H), 3.41 (m, 1H), 3.35 (m, 1H), 3.22 (m, 1H), 3.12 (m, 1H), 2.42 (s, 3H), 2.40 (s, 3H), 1.04 (d, J=7.2 Hz, 3H).

### EXAMPLE 3

### 3-({5-[5-Fluoro-2-oxo-1,2-dihydro-indol-(3Z)-ylidenemethyl]-2,4-dimethyl-1H-pyrrole-3-arbonyl}-amino)-butyric acid

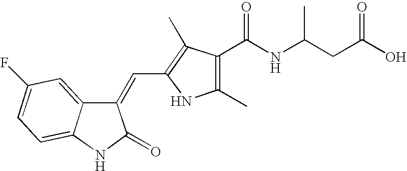

Preparative HPLC gave 14 mg of the title compound (56%) from 28 mg starting material (the active ester). LC-MS: single peak at 254 nm, MH⁺ calcd. for C₂₀H₂₀FN₃O₄: 386, obtained: 386. ¹H-NMR (DMSO-d₆, 400 MHz), δ 13.66 (s, 1H), 12.21 (s, 1H), 10.89 (s, 1H), 7.76 (dd, J=2.4 Hz, J=9.6 Hz, 1H), 7.71 (s, 1H), 7.57 (d, J=8.4 Hz, 1H), 6.92 (m, 1H), 6.83 (dd, J 4.8 Hz, J=8.4 Hz, 1H), 4.29 (m, 1H), 4.05 (m, 1H), 3.31 (d, J=9.6 Hz, 2H), 2.41 (s, 3H), 2.38 (s, 3H), 1.17 (d, J=6.8 Hz, 3H).

### EXAMPLE 4

### 5-[5-Fluoro-2-oxo-1,2-dihydro-indol-(3Z)-ylidenemethyl]-2,4-dimethyl-1H-pyrrole-3-carboxylic acid (2-dimethylcarbamoyl-1-methyl-ethyl)-amide

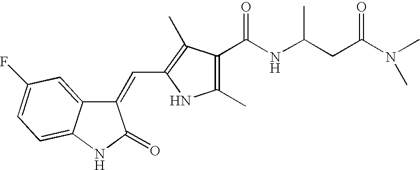

Preparative HPLC gave 42 mg of the title compound (78%) from 58 mg starting material (the active ester). LC-MS: single peak at 254 nm, MH⁺ calcd. for C₂₂H₂₅FN₄O₃: 413, obtained: 413. ¹H-NMR (DMSO-d₆, 400 MHz), δ 13.66 (s, 1H), 10.87 (s, 1H), 7.75 (dd, J=2.4 Hz, J=9.6 Hz, 1H), 7.70 (s, 1H), 7.55 (d, J=8.0 Hz, 1H), 6.92 (m, 1H), 6.82 (dd, J=4.8 Hz, J=8.4 Hz, 1H), 4.29 (m, 1H), 3.01 (s, 3H), 2.82 (s, 3H), 2.58 (m, 1H), 2.42 (m, 1H), 2.41 (S, 3H), 2.39 (s, 3H), 1.18 (d, J=6.8 Hz, 3H).

### EXAMPLE 5

### 5-[5-Fluoro-2-oxo-1,2-dihydro-indol-(3Z)-ylidenemethyl]-2,4-dimethyl-1H-pyrrole-3-carboxylic acid (1-methyl-3-(morpholin-4-yl)-3-oxo-propyl)-amide

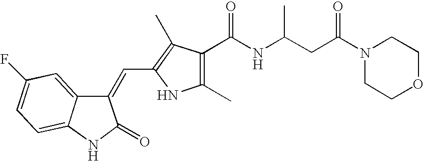

Preparative HPLC gave 43 mg of the title compound (73%) from 48 mg starting material (the active ester). LC-MS: single peak at 254 nm, MH⁺ calcd. for C₂₄H₂₇FN₄O₄: 455, obtained: 455. ¹H-NMR (DMSO-d₆, 400 MHz), δ 13.59 (s, 1H), 10.79 (s, 1H), 7.67 (dd, J=2.4 Hz, J=9.6 Hz, 1H), 7.63 (s, 1H), 7.47 (d, J=7.6 Hz, 1H), 6.85 (m, 1H), 6.76 (dd, J=4.8 Hz, J=8.4 Hz, 1H), 4.23 (m, 1H), 3.60-3.30 (m, 1OH), 2.35 (s, 3H), 2.32 (s, 3H), 1.11 (d, J=6.8 Hz, 3H).

### EXAMPLE 6

### 5-[5-Fluoro-2-oxo-1,2-dihydro-indol-(3Z)-ylidenemethyl]-2,4-dimethyl-1H-pyrrole-3-carboxylic acid ((S)-1-dimethylcarbamoyl-2-hydroxy-ethyl)-amide

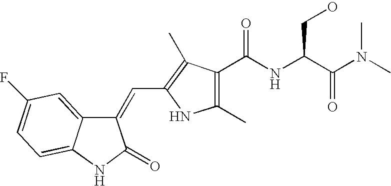

Preparative HPLC gave 42 mg of the title compound (84%) from 50 mg starting material (the active ester). LC-MS: single peak at 254 nm, MH⁺ calcd. for C₂₁H₂₃FN₄O₄: 415, obtained: 415. ¹H-NMR (DMSO-d₆, 400 MHz), δ 13.71 (s, 1H), 10.91 (s, 1H), 7.76 (dd, J=2.4 Hz, J=9.6 Hz, 1H), 7.72 (s, 1H), 7.56 (d, J=8.0 Hz, 1H), 6.92 (m, 1H), 6.84 (s, 1H), 6.83 (dd, J=4.8 Hz, J=8.4 Hz, 1H), 4.97 (m, 1H), 3.67 (m, 1H), 3.56 (m, 1H), 3.11 (s, 3H), 2.87 (s, 3H), 2.45 (s, 3H), 2.43 (s, 3H).

### EXAMPLE 7

### 5-[5-Fluoro-2oxo-1,2-dihydro-indol-(3Z)-ylidenemethyl]-2,4-dimethyl-1H-pyrrole-3-carboxylic acid ((S)-1-hydroxymethyl-2-(morpholin4-yl)-2-oxo-ethyl)-amide

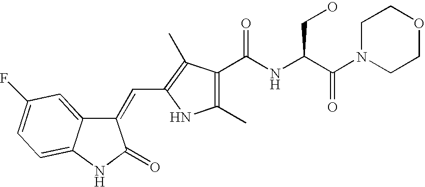

Preparative HPLC gave 51 mg of the title compound (93%) from 50 mg starting material (the active ester). LC-MS: single peak at 254 nm, MH⁺ calcd. for C₂₃H₂₅FN₄O₅: 457, obtained: 457. ¹H-NMR (DMSO-d₆, 400 MHz), δ 13.71 (s, 1H), 10.90 (s, 1H), 7.77 (dd, J=2.4 Hz, J=9.6 Hz, 1H), 7.73 (s, 1H), 7.63 (d, J=8.0 Hz, 1H), 6.94 (m, 1H), 6.83 (dd, J=4.8 Hz, J=8.4 Hz, 1H), 4.97 (m, 1H), 3.80-3.40 (m, ₁ 1H), 2.45 (s, 3H), 2.43 (s, 3H).

### EXAMPLE 8

### 5-[5-Fluoro-2-oxo-1,2-dihydro-indol-(3Z)-ylidenemethyl]-2,4-dimethyl-1H-pyrrole-3-carboxylic acid ((R)-1-dimethylcarbamoyl-2-hydroxy-ethyl)-amide

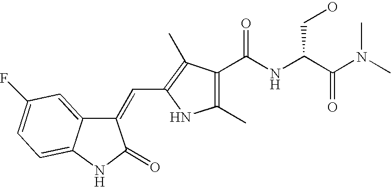

Preparative HPLC gave 40 mg of the title compound (64%) from 63 mg starting material (the active ester). LC-MS: single peak at 254 nm, MH⁺ calcd. for C₂₁H₂₃FN₄O₄: 415, obtained: 415. ¹H-NMR (DMSO-d₆, 400 MHz), δ 13.71 (s, 1H), 10.91 (s, 1H), 7.77 (dd., J=2.4 Hz, J=9.6 Hz, 1H), 7.72 (s, 1H), 7.55 (d, J=7.6 Hz, 1H), 6.93 (m, 1H), 6.84 (dd, J=4.8 Hz, J=8.4 Hz, 1H), 4.98 (dd, J=6.0Hz, J=14.0Hz, 1H), 3.67 (dd, J=6.4 Hz, J=14.8Hz, 1H), 3.58 (dd, J=6.4 Hz, J=14.4 Hz, 1H), 3.11 (s, 3H), 2.87 (s, 3H), 2.46 (s, 3H), 2.44 (s, 3H).

### EXAMPLE 9

### 5-[5-Fluoro-2-oxo-1,2-dihydro-indol-(3Z)-ylidenemethyl]-2,4-dimethyl-1H-pyrrole-3-carboxylic acid ((R)-1-hydroxymethyl-2-(morpholin4-yl)-2-oxo-ethyl)-amide

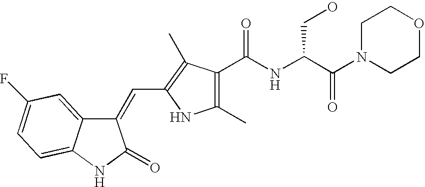

Preparative HPLC gave 32 mg of the title compound (47%) from 63 mg starting material (the active ester). LC-MS: single peak at 254 nm, MH⁺ calcd. for C₂₃H₂₅FN₄O₅: 457, obtained: 457. ¹H-NMR (DMSO-d₆, 400 MHz), δ 13.71 (s, 1H), 10.90 (s, 1H), 7.76 (dd, J=2.4 Hz, 9.6 Hz, 1H), 7.72 (s, 1H), 7.63 (d, J=8.0 Hz, 1H), 6.92 (m, 1H), 6.83 (dd, J=4.8 Hz, 8.4 Hz, 1H), 4.96 (dd, J=6.4 Hz, J=14.4 Hz, 1H), 3.74 (dd, J=6.4 Hz, J=14.4 Hz, 1H), 3.65-3.30 (m, 9H), 2.46 (s, 3H), 2.43 (s, 3H).

### EXAMPLE 10

### (S)-2-({5-[5-Fluoro-2-oxo-1,2-dihydro-indol-(3Z)-ylidenemethyl]-2,4-dimethyl-1H-pyrrole-3-carbonyl}-amino)-N*1*,N*1*,N*4*,N*4*-tetramethyl-succinamide

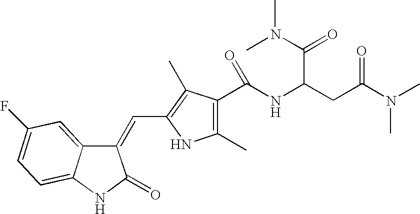

Preparative HPLC gave 30 mg of the title compound (73%) from 42 mg starting material (the active ester). LC-MS: single peak at 254 nm, MH⁺ calcd. for C₂₄H₂₈FN₅O₄: 470, obtained: 470. ¹H-NMR (DMSO-d₅, 400 MHz), δ 13.69 (s, 1H), 10.89 (s, 1H), 7.95 (d, J=8.8 Hz, 1H), 7.75 (dd, J=2.0 Hz, 9.2 Hz, 1H), 7.70 (s, 1H), 6.93 (m, 1H), 6.83 (dd, J=4.8 Hz, 8.4 Hz, 1H), 5.26 (m, 1H), 3.08 (s, 3H), 2.98 (s, 3H), 2.84 (s, 3H), 2.80 (s, 3H), 2.55 (m, 2H), 2.40 (s, 3H), 2.37 (s, 3H).

### EXAMPLE 11

### 5-[5-Fluoro-2-oxo-1,2-dihydro-indol-(3Z)-ylidenemethyl]-2,4-dimethyl-1H-pyrrole-3-carboxylic acid [(S)-1-(morpholine-4-carbonyl)-3-(morpholin-4-yl)-3-oxo-propyl]-amide

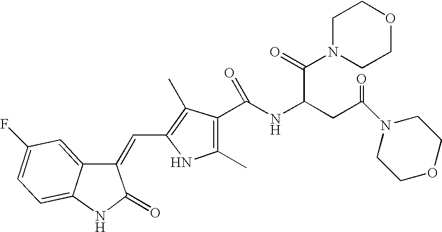

Preparative HPLC gave 70 mg of the title compound (97%) from 56 mg starting material (the active ester). LC-MS: single peak at 254 nm, MH⁺ calcd. for C₂₈H₃₂FN₅O₆: 554, obtained: 554. ¹H-NMR (DMSO-d₆, 400 MHz), δ 13.68 (s, 1H), 10.91 (s, 1H), 8.08 (d, J=8.8 Hz, 1H), 7.76 (dd, J=2.4 Hz, 9.2 Hz, 1H), 7.71 (s, 1H), 6.93 (m, 1H), 6.83 (dd, J=4.8 Hz, 8.4 Hz, 1H), 5.28 (m, 1H), 3.75 (m, 2H), 3.70-2.50 (m, 16H), 2.41 (s, 3H), 2.38 (s, 3H).

### EXAMPLE 12

### (S)-2-({5-[5-Fluoro-2-oxo-1,2-dihydro-indol-(3Z)-ylidenemethyl]-2,4-dimethyl-1H-pyrrole-3-carbonyl}-amino)-pentanedioic acid bis-dimethylamide

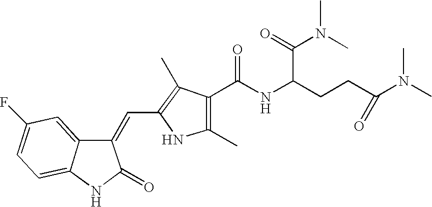

Preparative HPLC gave 60 mg of the title compound (78%) from 75 mg starting material (the active ester). LC-MS: single peak at 254 nm, MH⁺ calcd. for C₂₅H₃₀FN₅O₄: 484, obtained: 484. ¹H-NMR (DMSO-d₆, 400 MHz), δ 13.69 (s, 1H), 10.88 (s, 1H), 7.75 (dd, J=2.4 Hz, 9.6 Hz, 1H), 7.71 (s, 1H), 7.70 (d, J=8.0 Hz, 1H), 6.93 (m, 1H), 6.84 (dd, J=4.8 Hz, 8.4 Hz, 1H), 4.88 (m, 1H), 3.13 (s, 3H), 2.94 (s, 3H), 2.86 (s, 3H), 2.82 (s, 3H), 2.44 (s, 3H), 2.42 (s, 3H), 2.34 (m, 2H), 1.95 (m, 1H), 1.74 (m, 1H).

### EXAMPLE 13

### 5-[5-Fluoro-2-oxo-1,2-dihydro-indol-(3Z)-ylidenemethyl]-2,4-dimethyl-1H-pyrrole-3-carboxylic acid [(S)-1-(morpholine4-carbonyl)4-(morpholin4-yl)-4-oxo-butyl]-amide

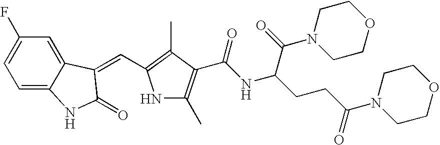

Preparative HPLC gave 82 mg of the title compound (94%) from 75 mg starting material (the active ester). LC-MS: single peak at 254 nm, MH⁺ calcd. for C₂₉H₃₄FN₅O₆: 568, obtained: 568. ¹H-NMR (DMSO-d₆, 400 MHz), δ 13.70 (s, 1H), 10.91 (s, 1H), 8.30 (m, 1H), 7.78 (m, 1H), 7.72 (s, 1H), 6.92 (m, 1H), 6.84 (m, 1H), 4.90 (m, 1H), 3.80-3.35 (m, 9H), 3.13 (m, 7H), 2.45 (s, 3H), 2.43 (s, 3H), 2.56-2.35 (m, 2H), 1.97 (m, 1H), 1.76 (m, 1H).

### EXAMPLE 14

### (S)-4-Dimethylcarbamoyl-2-({5-[5-fluoro-2-oxo-1,2-dihydro-indol-(3Z)-ylidenemethyl]-2,4-dimethyl-1H-pyrrole-3-carbonyl}-amino)-butyric acid

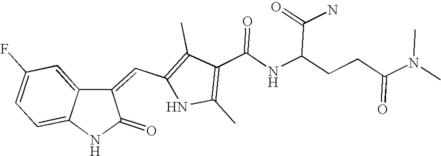

Preparative HPLC gave 44 mg of the title compound (81%) from 50 mg starting material (the active ester). LC-MS: single peak at 254 nm, MH⁺ calcd. for C₂₃H₂₅FN₄O₅: 457, obtained: 457.

### EXAMPLE 15

### (S)-2-({5-[5-Fluoro-2-oxo-1,2-dihydro-indol-(3Z)-ylidenemethyl]-2,4-dimethyl-1H-pyrrole-3-carbonyl}-amino)-5-morpholin-4-yl-5-oxo-pentanoic acid

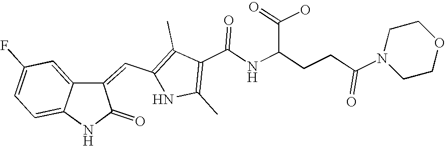

Preparative HPLC gave 40 mg of the title compound (67%) from 50 mg starting material (the active ester). LC-MS: single peak at 254 nm, MH⁺ calcd. for C₂₅H₂₇FN₄O₆: 499, obtained: 499. ¹H-NMR (DMSO-d₆, 400 MHz), δ 13.69 (s, 1H), 12.55 (s, 1H), 10.89 (s, 1H), 7.88 (d, J=8.0 Hz, 1H), 7.75 (dd, J=2.4 Hz, J=9.6 Hz, 1H), 7.72 (s, 1H), 6.93 (m, 1H), 6.84 (dd, J=4.8 Hz, 8.4 Hz, 1H), 4.36 (m, 1H), 3.53 (m, 4H), 3.42 (m, 4H), 3.31 (m, 2H), 2.44 (s, 3H), 2.42 (s, 3H), 2.08 (m, 1H), 1.93 (m, 1H).

### EXAMPLE 16

### (R)-2-({5-[5-Fluoro-2-oxo-1,2-dihydro-indol-(3Z)-ylidenemethyl]-2,4-dimethyl-1H-pyrrole-3-carbonyl}-amino)-5-morpholin-4-yl-5-oxo-pentanoic acid

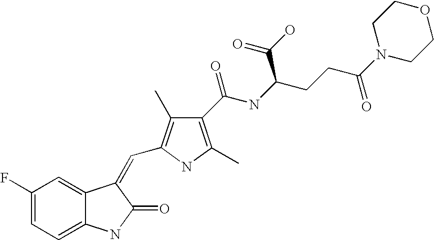

Preparative HPLC gave 37 mg of the title compound (84%) from 37 mg starting material (the active ester). LC-MS: single peak at 254 nm, MH⁺ calcd. for C₂₅H₂₇FN₄O₆: 499, obtained: 499. ¹H-NMR (DMSO-d₆, 400 MHz), δ 13.69 (s, 1H), 12.57 (s, 1H), 10.90 (s, 1H), 7.88 (d, J=8.0 Hz, 1H), 7.76 (dd, J=2.8 Hz, 9.2 Hz, 1H), 7.72 (s, 1H), 6.92 (m, 1H), 6.84 (dd, J=4.8 Hz, 8.4 Hz, 1H), 4.37 (m, 1H), 3.53 (m, 3H), 3.43 (m, 4H), 3.31 (m, 3H), 2.45 (s, 3H), 2.42 (s, 3H), 2.08 (m, 1H), 1.93 (m, 1H).

### EXAMPLE 17

### (R)-2-({5-[5-Fluoro-2-oxo-1,2-dihydro-indol-(3Z)-ylidenemethyl]-2,4-dimethyl-1H-pyrrole-3-carbonyl}-amino)-pentanedioic acid bis-dimethylamide

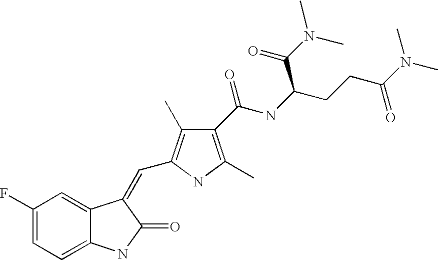

Preparative HPLC gave 28 mg of the title compound (41%) from 60 mg of starting material (the active ester). LC-MS: single peak at 254 nm, MH⁺ calcd. for C₂₅H₃₀FN₅O₄: 484, obtained: 484. ¹H-NMR (DMSO-d₆, 400 MHz), δ 13.69 (s, 1H), 10.90 (s, 1H), 7.76 (dd, J=2.4 Hz, 9.6 Hz, 1H), 7.73 (d, J=8.0 Hz, 1H), 7.72 (s, 1H), 6.93 (m, 1H), 6.84 (dd, J=4.8 Hz, 8.4 Hz, 1H), 4.88 (m, 1H), 3.13 (s, 3H), 2.93 (s, 3H), 2.86 (s, 3H), 2.82 (s, 3H), 2.44 (s, 3H), 2.42 (s, 3H), 2.50-2.30 (m, 2H), 1.95 (m, 1H), 1.74 (m, 1H).

### EXAMPLE 18

### 5-[5-Fluoro-2-oxo-1,2-dihydro-indol-(3Z)-ylidenemethyl]-2,4-dimethyl-1H-pyrrole-3-carboxylic acid [(R)-1-(morpholine-4-carbonyl)-4-(morpholin-4-yl)-4-oxo-butyl]-amide

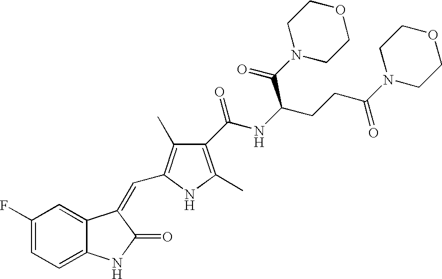

Preparative HPLC gave 30 mg of the title compound (38%) from 60 mg of starting material (the active ester). LC-MS: single peak at 254 nm, MH⁺ calcd. for C₂₉H₃₄FN₅O₆: 568, obtained: 568. ¹H-NMR (DMSO-d₆, 400 MHz), δ 13.70 (s, 1H), 10.91 (s, 1H), 7.77 (m, 2H), 7.72 (s, 1H), 6.93 (m, 1H), 6.83 (m, 1H), 4.91 (m, 1H), 3.90-3.35 (m, 16H), 2.45 (s, 3H), 2.42 (s, 3H), 2.50-2.30 (m, 2H), 1.98 (m, 1H), 1.77 (m, 1H).

### EXAMPLE 19

### 5-[5-Fluoro-2-oxo-1,2-dihydro-indol-(3Z)-ylidenemethyl]-2,4-dimethyl-1H-pyrrole-3-carboxylic acid ((1S,2S)-1-dimethylcarbamoyl-2-hydroxy-propyl)-amide

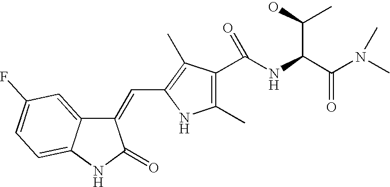

Preparative HPLC gave 84 mg of the title compound (67%) from 122 mg starting material (the active ester). LC-MS: single peak at 254 nm, MH⁺ calcd. for C₂₂H₂₅FN₄O₄: 429, obtained: 429. ¹H-NMR (DMSO-d₆, 400 MHz), δ 13.69 (s, 1H), 10.89 (s, 1H), 7.75 (m, 1H), 7.70 (s, 1H), 7.61 (d, J=8.8 Hz, 1H), 6.92 (m, 1H), 6.83 (dd, J=4.8 Hz, 8.4 Hz, 1H), 4.81 (t, J=4.4 Hz, 1H), 3.90 (m, 1H), 3.12 (s, 3H), 2.86 (s, 3H), 2.42 (s, 3H), 2.39 (s, 3H), 1.12 (d, J=4.8 Hz, 3H).

### EXAMPLE 20

### 5-[5-Fluoro-2-oxo-1,2-dihydro-indol-(3Z)-ylidenemethyl]-2,4-dimethyl-1H-pyrrole-3-carboxylic acid ((1R,2R)-1-dimethylcarbamoyl-2-hydroxy-propyl)-amide

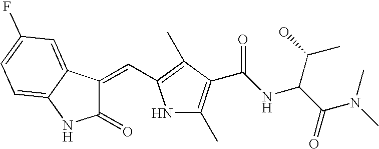

Preparative HPLC gave 78 mg of the title compound (62%) from 122 mg starting material (the active ester). LC-MS: single peak at 254 nm, MH⁺ calcd. for C₂₂H₂₅FN₄O₄: 429, obtained: 429. ¹H-NMR (DMSO-d₆, 400 MHz), δ 13.70 (s, 1H), 10.90 (s, 1H), 7.77 (m, 1H), 7.71 (s, 1H), 7.62 (d, J=8.4 Hz, 1H), 6.93 (m, 1H), 6.84 (dd, J=4.8 Hz, 8.4 Hz, 1H), 4.82 (t, J=8.0 Hz, 1H), 3.92 (m, 1H), 3.13 (s, 3H), 2.87 (s, 3H), 2.43 (s, 3H), 2.40 (s, 3H), 1.15 (d, J=2.8 Hz, 3H).

### EXAMPLE 21

### 5-[5-Fluoro-2-oxo-1,2-dihydro-indol-(3Z)-ylidenemethyl]-2,4-dimethyl-1H-pyrrole-3-carboxylic acid ((1R,2S)-1-dimethylcarbamoyl-2-hydroxy-propyl)-amide

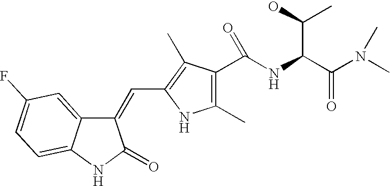

Preparative HPLC gave 90 mg of the title compound (72%) from 122 mg starting material (the active ester). LC-MS: single peak at 254 nm, MH⁺ calcd. for C₂₂H₂₅FN₄O₄: 429, obtained: 429. ¹H-NMR (DMSO-d₆, 400 MHz), δ 13.73 (s, 1H), 10.91 (s, 1H), 7.77 (dd, J=2.4 Hz, 6.4 Hz, 1H), 7.73 (s, 1H), 7.29 (d, J=8.0 Hz, 1H), 6.93 (m, 1H), 6.84 (dd, J=4.8 Hz, 8.4 Hz, 1H), 4.85 (m, 1H), 3.97 (m, 1H), 3.12 (s, 3H), 2.87 (s, 3H), 2.43 (s, 3H), 2.41 (s, 3H), 1.10 (d, J=5.6 Hz, 3H).

### EXAMPLE 22

### 5-[5-Fluoro-2-oxo-1,2-dihydro-indol-(3Z)-ylidenemethyl]-2,4-dimethyl-1H-pyrrole-3-carboxylic acid ((1S,2R)-1-dimethylcarbamoyl-2-hydroxy-propyl)-amide

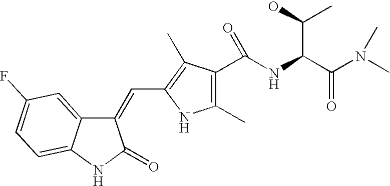

Preparative HPLC gave 90 mg of the title compound (72%) from 122 mg starting material (the active ester). LC-MS: single peak at 254 nm, MH⁺ calcd. for C₂₂H₂₅FN₄O₄: 429, obtained: 429. ¹H-NMR (DMSO-d₆, 400 MHz), δ 13.73 (s, 1H), 10.91 (s, 1H), 7.76 (dd, J=2.8 Hz, 6.8 Hz, 1H), 7.73 (s, 1H), 7.30 (d, J=8.0 Hz, 1H), 6.93 (m, 1H), 6.84 (dd, J=4.8 Hz, 8.4 Hz, 1H), 4.85 (m, 1H), 3.98 (m, 1H), 3.12 (s, 3H), 2.86 (s, 3H), 2.48 (s, 3H), 2.45 (s, 3H), 1.10 (d, J=6.0 Hz, 3H).

### EXAMPLE 23

### 5-[5-Fluoro-2-oxo-1,2-dihydro-indol-(3Z)-ylidenemethyl]-2,4-dimethyl-1H-pyrrole-3-carboxylic acid dimethylcarbamoylmethyl-amide

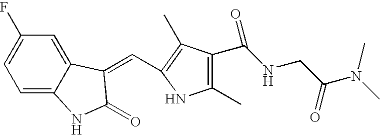

Preparative HPLC gave 27 mg of the title compound (46%) from 66 mg starting material (the active ester). LC-MS: single peak at 254 nm, MH⁺ calcd. for C₂₀H₂₁FN₄O₃: 385, obtained: 385. ¹H-NMR (DMSO-d₆, 400 MHz), δ 13.71 (s, 1H), 10.90 (s, 1H), 7.76 (dd, J=2.4 Hz, J=9.6 Hz, 1H), 7.73 (s, 1H), 7.55 (t, J=5.6 Hz, 1H), 6.93 (rm, 1H), 6.84 (dd, J=4.4 Hz, 8.4 Hz, 1H), 4.08 (d, J=5.6 Hz, 2H), 3.00 (s, 3H), 2.87 (s, 3H), 2.49 (s, 3H), 2.46 (s, 3H).

### VEGFR Biochemical Assay

The compounds were assayed for biochemical activity by Upstate Ltd at Dundee, United Kingdom, according to the following procedure. In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [γ-³³P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Cellular Assay: HUVEC: VEGF Induced Proliferation

The compounds were assayed for cellular activity in the VEGF induced proliferation of HUVEC cells. HUVEC cells (Cambrex, CC-2517) were maintained in EGM (Cambrex, CC-3124) at 37° C. and 5% CO₂. HUVEC cells were plated at a density 5000 cells/well (96 well plate) in EGM. Following cell attachment (1 hour) the EGM-medium was replaced by EBM (Cambrex, CC-3129)+0.1% FBS (ATTC, 30-2020) and the cells were incubated for 20 hours at 37° C. The medium was replaced by EBM +1% FBS, the compounds were serial diluted in DMSO and added to the cells to a final concentration of 0-5,000 nM and 1% DMSO. Following a 1 hour pre-incubation at 37° C. cells were stimulated with 10 ng/ml VEGF (Sigma, V7259) and incubated for 45 hours at 37° C. Cell proliferation was measured by BrdU DNA incorporation for 4 hours and BrdU label was quantitated by ELISA (Roche kit, 16472229) using 1M H₂SO₄ to stop the reaction. Absorbance was measured at 450 nm using a reference wavelength at 690 nm.

1-31. (canceled)

32. A compound represented by Formula I as follows: 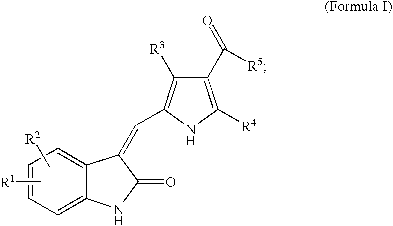
wherein:
R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, and substituted or unsubstituted (C6-C10) aryl;
R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, and (C6-C10) arylamino;
R³ is selected from the group consisting of hydrogen, (C1-C6) alkyl, (C6-C10) aryl, (C5-C10) heteroaryl, and amide;
R⁴ is selected from the group consisting of hydrogen and (C1-C6) alkyl; and
R⁵ is an alpha or beta amino acid group or an alpha or beta amino amide group connected to a carbonyl of the compound of Formula (I) through a respective alpha or beta amino group to form an amide bond;
or a pharmaceutically acceptable salt or prodrug thereof.

33. A compound according to claim 32 wherein R⁵ is represented by the following structure: 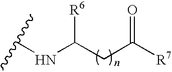
wherein:
R⁶ is a side chain of a naturally or unnaturally occurring amino acid or its corresponding amide derivative thereof, the amide derivative having an amide nitrogen represented by NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, and (C3-C8) cycloalkyl carboxylic acid, or R⁸ and R⁹ together with N to which R⁸ and R⁹ are bonded form a (C5-C8) heterocyclic ring either unsubstituted or substituted with one or more hydroxyls, ketones, ethers, and carboxylic acids; wherein a wavy line indicates a point of attachment to the carbonyl of the compound of Formula I;
R⁷ is selected from the group consisting of hydroxy, (C1-C6) O-alkyl, (C3-C8) O-cycloalkyl, and NR⁸R⁹; and
n is 0 or 1.

34. A compound according to claim 33 wherein R⁵ is an alpha amino acid where the alpha amino group is connected to the carbonyl of Formula I to form an amide bond.

35. A compound according to claim 33 wherein R⁵ is an alpha amino amide where the alpha amino group is connected to the carbonyl of Formula I to form an amide bond.

36. A compound according to claim 33 wherein R⁵ is a beta amino acid where the beta amino group is connected to the carbonyl of Formula I to form an amide bond.

37. A compound according to claim 33 wherein R⁵ is a beta amino amide where the beta amino group is connected to the carbonyl of Formula I to form an amide bond.

38. A compound according to claim 34 having the following structure:

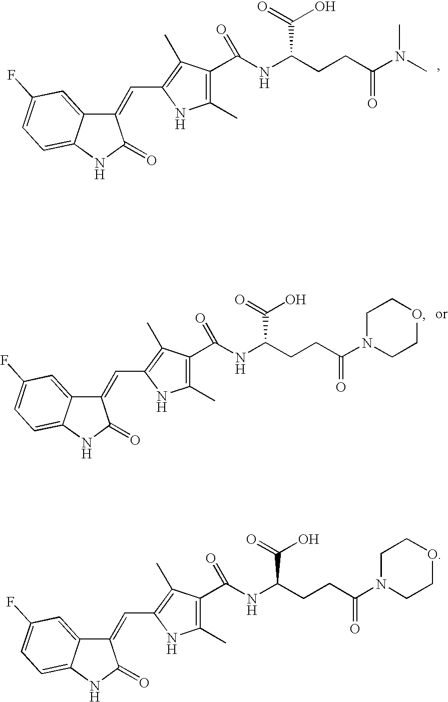

39. A compound according to claim 35 having the following structure:

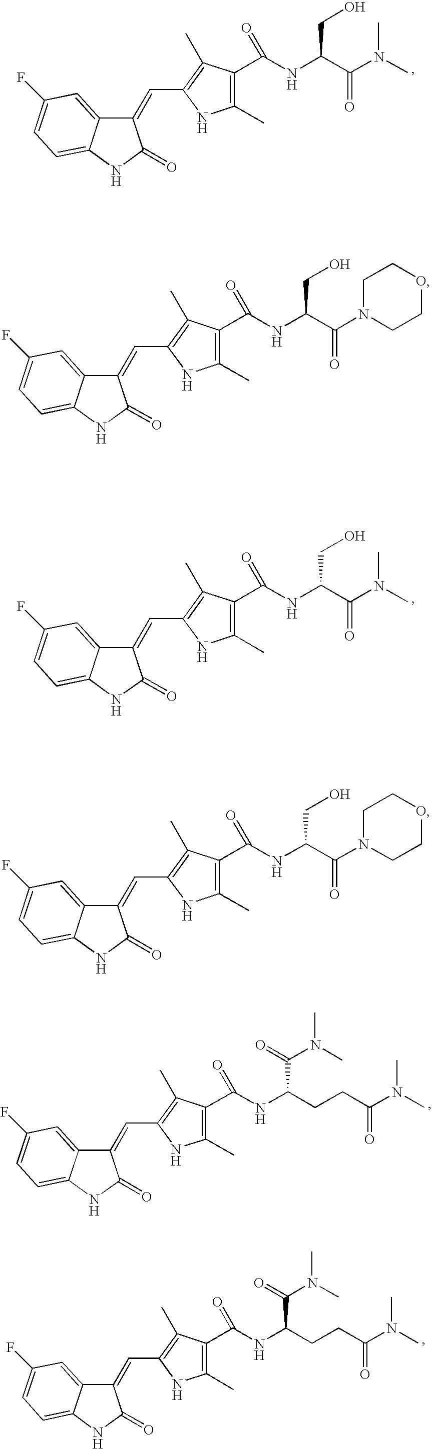

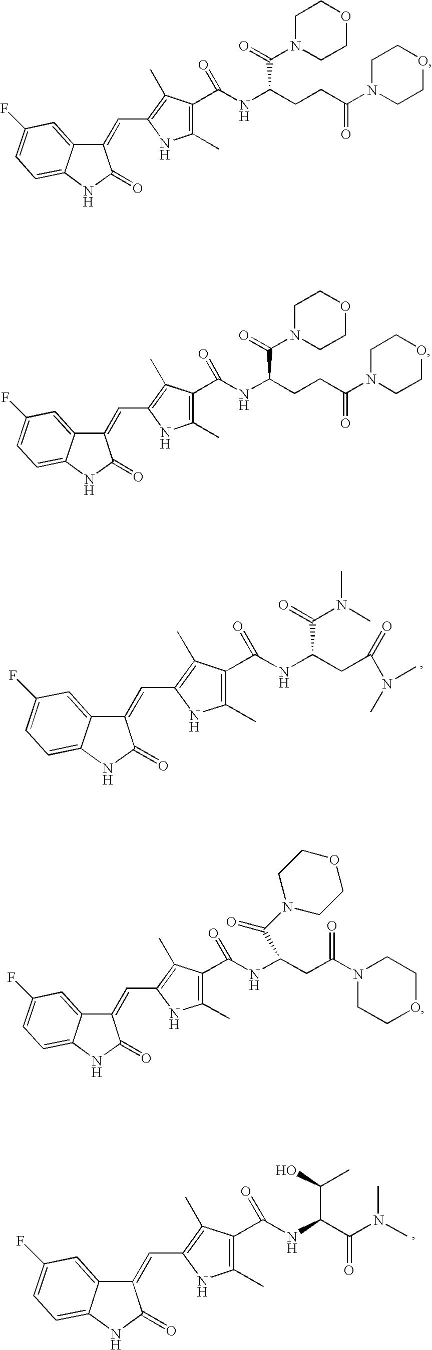

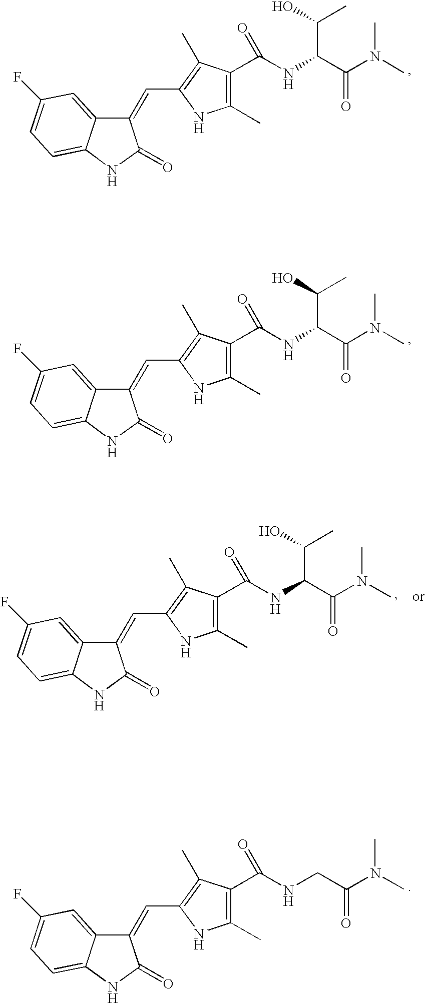

40. A compound according to claim 36 having the following structure:

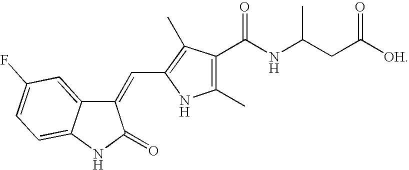

41. A compound according to claim 37 having the following structure:

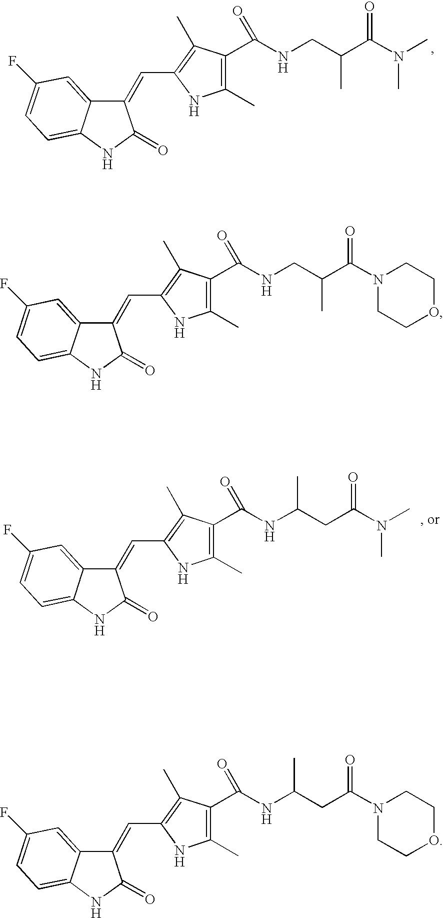

42. A method for the modulation of the catalytic activity of a protein kinase with a compound or salt thereof of any one of claims 32-41.

43. The method of claim 42, wherein said protein kinase is selected from the group of receptors consisting of VEGFR and PDGFR.

44. The method of claim 42 wherein the compound or salt thereof acts as a prodrug.
